(19) United States
(12) Patent Application Publication
(10) Pub. No.: US 2006/0009492 A1
(43) Pub. Date: Jan. 12, 2006

(54) Cancer treatment with compounds inhibiting PKC alpha

(76) Inventor: Zhimin Lu, Pearland, TX (US)

Correspondence Address: Robert E. Bushnell, Suite 300, 1522 K Street, N.W., Washington, DC, 20005, US

(21) Appl. No.: 11/175,315

(22) Filed: Jul. 7, 2005

(30) Foreign Application Priority Data
Jul. 9, 2004 (CN) 2004100627688

(51) Int. Cl. A61K 31/445; A61K 31/454

(52) U.S. Cl. 514/320

(57) ABSTRACT

A chemotherapeutic cancer treatment with Gö7874, Gö6850, Gö7612, Gö7852, Ro31-7549, Ro31-8425, Ro31-8220, Ro31-0432 and NGIC-1 or chemical compounds with similar structures is administered to a mammal for the treatment of the cancer. The chemical compound is targeted to PKC α activity. Experiments indicate that Gö7874, Gö6850, Gö7612, Gö7852, Ro31-7549, Ro31-8425, Ro31-8220, Ro31-0432 and NGIC-1 and similar compounds to be effective for the treatment of breast cancer, leukemia, lung cancer, skin cancer, prostate cancer, liver cancer, brain tumor, cervical cancer, and cancers located in the digestive tract including gastric cancer and colorectal cancers. These treatments may be accomplished utilizing Gö7874, Gö6850, Gö7612, Gö7852, Ro31-7549, Ro31-8425, Ro31-8220, Ro31-0432 and NGIC-1 and chemical compounds with similar structures alone or in combination with prior art chemotherapy agents or with radiation therapy. In a preferred embodiment Gö7874, Gö6850, Gö7612, Gö7852, Ro31-7549, Ro31-8425, Ro31-8220, Ro31-0432 and NGIC-1 chemical compounds with similar structures is used for the treatment of cancer as a preventative drug by preventing cancer cell formation.

### CLAIM OF PRIORITY

This application makes reference to, incorporates the same herein, and claims all benefits accruing under 35 U.S.C. §119 from an application earlier filed in the Chinese Patent Office on 9 Jul. 2004 and there duly assigned Serial No. 2004100627688.

### BACKGROUND OF THE INVENTION

1. Field of the Invention

2. Description of the Related Art

Researchers have recognized that a family of enzymes known as protein kinase C enzymes is associated with a large number of cancers. This family includes at least eleven isoenzymes. A particular member of this family is identified as the protein kinase C alpha enzyme, abbreviated to PKC α.

Researches have reported increases in PKC α activity in human breast tumors (NG et al., Science. 283:2085-2089) and significant increases in PKC α expression in prostate cancers (Cornford et al., Am. J. Pathol. 154: 137-144). Researchers have reported that PKC α is required for the metastasis of human melanoma (Dennis et al., Cancer Lett. 128:65-70) and that PKC α is related to the progression of brain tumors (Shen et al., Mol. Pharmacol. 55:396-402). Inhibition of PKC α with chemotherapeutic reagents or specially designed oligonucleotides can be used for cancer treatment.

Recently, Muller et al. were granted a patent, U.S. Pat. No. 5,744,460, which discloses a cancer treatment utilizing an antisense oligonucleotide targeted to PKC α combined with a chemotherapeutic agent. U.S. Pat. Nos. 5,882,927 and 5,885,970 issued to Bennett et al. also disclose antisense oligonuclotides targeted to PKC α. Several patents that disclose PKC inhibitors for treating cancers have been issued to Heath, Jr., et al. These include U.S. Pat. No. 5,843,935. U.S. Pat. No. 5,723,456 is an example of a treatment in which a PKC directed enzyme is proposed as a method for treating atherosclerotic disease states especially cardiovascular diseases associated with vascular endothelial cell dysfunction. U.S. Pat. No. 5,821,072 granted to Schwartz, et al. proposes the use of certain chemotherapeutic agents to attempt to reduce PKC activity. The disclosures of the above listed patents are incorporated herein by reference.

A chemical known as Gö6976 is known to be an inhibitor of PKC α. U.S. Pat. No. 6,303,646 and U.S. Pat. No. 6,696,478 disclose that Gö6976 can use as a cancer treating and prevention reagent.

### SUMMARY OF THE INVENTION

It is therefore an object of the present invention to provide a chemotherapeutic cancer treatment method.

It is a further object of the present invention to provide a chemotherapeutic cancer treatment in which chemical compounds are able to inhibit PKC α activity.

According to an aspect of the present invention, a method of treating or preventing a cancer, including: administering to a mammal a chemical targeted to PKC α, said chemical being a compound having one of the following formulae or its derivative:

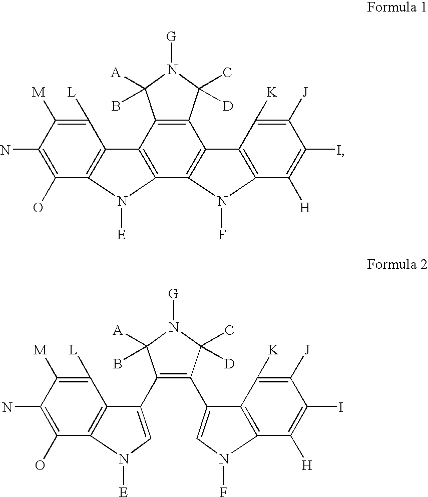

wherein each or a combination of two of A, B, C, D, E, F, G, H, I, J, K, L, M, N, and O is independently selected from the group consisting of hydrogen, oxygen, methyl, ethyl, propyl, isopropyl, carboxymethyl, 2-carboxyethyl, 3-carboxypropyl, a straight or branched alkyl group, a straight or branched substituted alkyl group, a straight or branched azidoalkyl, carboxyalkyl, amidinothioalkyl, amidinoalkyl, (2-nitroguanidino) alkyl group, cyanoalkyl, and —(CH₂)₂—CO—NX wherein X is hydrogen, alkyl, or benzyl, and the compound represented by Formula 1 is not 12-(2-Cyanoethyl)-6,7,12,13-tetrahydro-13-methyl-5-oxo-5H-indolo[2,3-a]pyrrolo[3,4-c]carbazole, 12-(3-Aminopropyl)-5,6,7,12,13-pentahydro-indolo[2,3,-a] pyrrolo [3,4-c] carbazole, or 12-(3-Aminopropyl)-5,6,7,12,13-pentahydro-indolo[2,3,-a] pyrrolo [3,4-c] carbazole hydrochloride; and

monitoring said mammal to determine state of said cancer.

It is preferred that the chemical is selected from a group consisting of Gö7874, Gö6850, Gö7612, Gö7852, Ro31-7549, Ro31-8425, Ro31-8220, Ro31-0432, NGIC-I, a derivative thereof, and a salt thereof.

### DETAILED DESCRIPTION OF PREFERRED EMBODIMENTS

The present invention provides a chemotherapeutic cancer treatment in which chemical compounds have the following formulas are administered to a mammal for the treatment of the cancer. The chemical compounds are able to inhibit PKC α activity. Experiments have shown that the compounds having these formulas such as Gö7874, Gö6850, Gö7612, Gö7852, Ro31-7549, Ro31-8425, Ro31-8220, Ro31-0432 and NGIC-1 are effective for the treatment of breast cancer, leukemia, lymphoma, lung cancer, skin cancer, prostate cancer, liver cancer, brain tumor, cervical cancer, pancreatic cancer, head and neck cancer, and cancers located in the digestive tract including gastric cancer and colorectal cancers. These treatments may be accomplished utilizing these compounds and compounds similar to them alone or in combination with the prior art chemotherapy agents or with radiation therapy. For example, a salt and/or a regioisomeric mixture of the compounds may be used alone or in combination with the prior art chemotherapy agents or with radiation therapy.

In a preferred embodiment these compound are used for the treatment of cancer as a preventative drug by preventing cancer cell formation. In the specification and the claims, the meaning of treating cancer includes this preventative treatment.

In a preferred embodiment the chemical targeted to PKC α is a chemical having the following formulae:

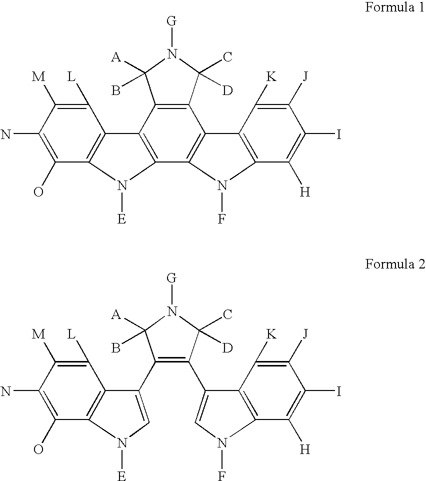

where each or a combination of the chemical groups of A, B, C, D, E, F, G, H, I, J, K, L, M, N, and O are preferably independently selected from the following group consisting of hydrogen, oxygen, methyl, ethyl, propyl, or isopropyl, carboxymethyl, 2-carboxyethyl, or 3-carboxypropyl, straight or branched alkyl, straight or branched substituted alkyl, a straight or branched azidoalkyl, carboxyalkyl, amidinothioalkyl, amidinoalkyl, (2-nitroguanidino) alkyl, cyanoalkyl, or —(CH₂)₂—CO—NX wherein X can be each independently hydrogen, alkyl or benzyl. The chemical groups for A, B, C, D, E, F, G, H, I, J, K, L, M, N, and O should be interpreted to be fit into the above chemical structure. For example, two neighboring groups may form a carbonyl group with the carbon in the above formula. More particularly, for example, A and B may be an oxygen bonded to the carbon by a double bond to form a carbonyl group, as shown in Formula III.

Preferred chemicals for use in accordance with the present invention are Gö7874, Gö6850, Gö7612, Gö7852, Ro31-7549, Ro31-8425, Ro31-8220, Ro31-0432 and NGIC-1.

(A) Gö7874: C₂₇H₂₆N₄O₄.HCl.
5,7(6H)-dione,13-[3-(dimethylamino)-2-hydroxypropyl]-12,13-dihydro-3-methoxy-12-methyl-5H-Indolo[2,3-a]pyrrolo[3,4-c]carbazole, monohydrochloride.

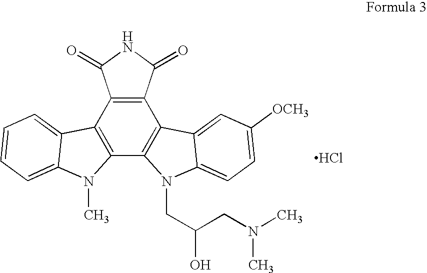

(B) Gö6850; C₂₅H₂₄N₄O₂. Bisindolylmaleimide I; GF 109203X;
2-[1-(3-Dimethylaminopropyl)-1H-indol-3-yl]-3-(1H-indol-3-yl)-maleimide.

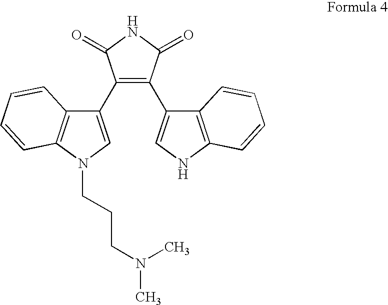

(C) NGIC-I; C₂₃H₁₆N₄O. Non-glycosidic Indolocarbazole I.
12-propanenitrile,5,6,7,13-tetrahydro-5-oxo-12H-Indolo[2,3-a]pyrrolo[3,4-c]carbazole.

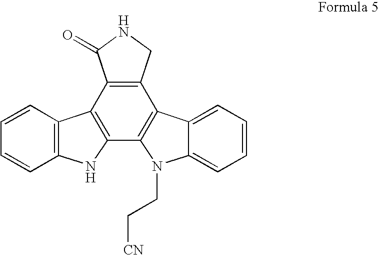

(D) Gö7852; 5-one, 12-[3-(dimethylamino)-2-hydroxypropyl]-6,7,12,13-tetrahydro-13-methyl-5H-Indolo[2,3-a]pyrrolo[3,4-c]carbazol.

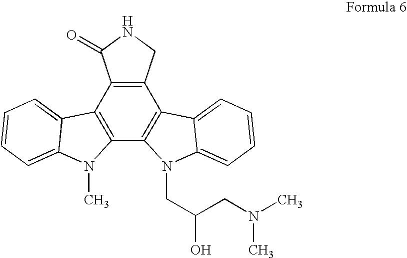

(E) Gö7612; 12-propanenitrile, 5,6,7,13-tetrahydro-9-methoxy-13-methyl-5,7-dioxo-12H-Indolo[2,3-a]pyrrolo[3,4-c]carbazole.

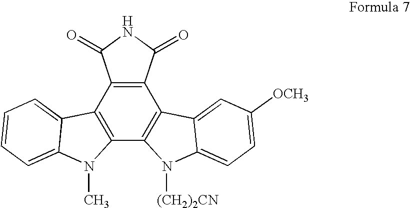

(F) Ro31-7549; C₂₄H₂₂N₄O₂C₂H₄O₂. Bisindolylmaleimide VIII, Acetate; 2-[1-3(Aminopropyl)indol-3-yl]-3(1-methyl-1H-indol-3-yl)maleimide, Acetate.

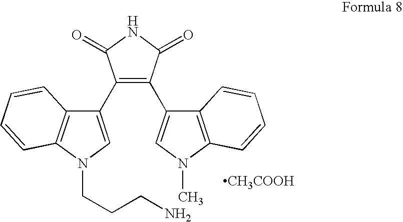

(G) Ro31-8220; C₂₅H₂₃N₅O₂S.CH₄O₃S. Bisindolylmaleimide IX, Methane sulfonate; 3-[1-[3-(Amidinothio)propyl-1H-indol-3-yl]-3-(1-methyl-1H-indol-3-yl)maleimide.

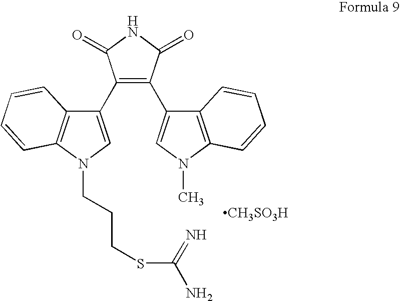

(H) Ro31-8425; C26H24N4O2.HCl. Bisindolylmaleimide X, HCl.

2-[8-(Aminomethyl)-6,7,8,9-tetrahydropyrido[1,2-a]indol-3-yl]-3-(1-methyl-1H-indol-3-yl)maleimide, HCl.

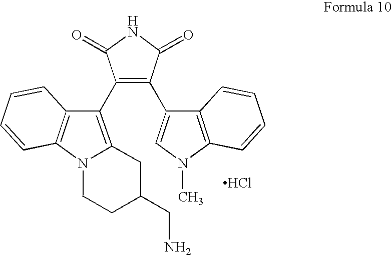

(I) Ro31-0432; C₂₈H₂₈N₄O₂HCl. Bisindolylmaleimide XI, HCl;

2-{8-[(Dimethylamino)methyl]-6,7,8,9-tetrahydropyrido[1,2-a]indol-3-yl}-3-(1-methyl-1H-indol-3-yl)maleimide, HCl. 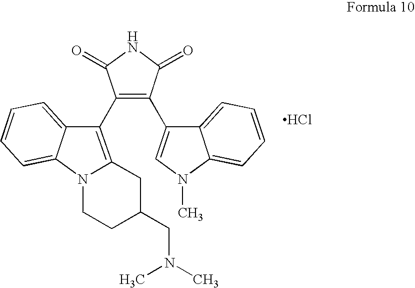
Cell Culture

The following cancer cells were maintained in Dulbecco's modified Eagle medium (DMEM) supplemented with 10 percent bovine calf serum.

PC3 human prostate cancer cells;

A549 human lung cancer cells;

HepG2 human liver cancer cells;

AGS human gastric cancer cells;

HT29 human colon cancer cells;

U251 human glioma cells;

Hela human cervical cancer cells;

MDA-MB-468 human breast cancer cells;

A431 human epidermoid carcinoma cells;

PANC-1 human pancreatic carcinoma cells;

Jurkat cells (a human acute T-cell leukemia cell line);

KB oral cancer cells;

K562 leukemia cells (human chronic myelogenous leukemia);

H460 human lung cancer cells; and

NBT-II human bladder cancer cells.

Cell Viability Assay

Cells were seeded for 24 hours. Then they were treated with PKC inhibitors or left untreated. The cells were then collected periodically and evaluated using trypan blue dye exclusion to assess viability using the method described by C Y Chen, et al., in J. Biol. Chem. 273:16700-16709.

Soft Agar/Cell Transformation Assay

1×10³ 3Y1 rat fibroblast cells overexpressing the epidermal growth factor receptor (EGFR) were suspended in top agar (consisting of 20 percent calf serum, 0.38 percent agar and the remainder DMEM) and overlaid onto bottom agar (DMEM, 20 percent calf serum and 0.7 percent agar) as reported by Sementchenko et al., in Onocgen 17:2883-2888.

Practice of the invention will be more understood from the following examples, which are presented herein for illustration only and should not be considered as limiting the invention in any way.

### EXAMPLES

### Example 1

### The Effect of Gö7874 on Cancer Cell Growth and Survival

The results of treatment of a variety of human cancer cells with Gö7874 are shown in Table 1. Cultures containing about 4×10⁴ cells were treated with 5 micromoles of Gö7874 (except that PC3 prostate cancer cells were treated with 10 micromoles) and the viable cells were counted at day 6. As shown in Table 1, while untreated cells proliferate and increase cell number by several folds, the cancer cells treated with Gö6976 were significantly reduced in number. PC3 human prostate cancer cells, MDA-MB-468 human breast cancer cells, H460 human lung cancer cells, HT29 human colon cancer cells, AGS human gastric cancer cells, HepG2 human liver cancer cells, PANC-1 human pancreatic carcinoma cells, A431 human epidermoid carcinoma cells, U251 human glioma cells, Hela human cervical cancer cells, KB oral cancer cells, and U-937 lymphoma cells all went to apoptosis after treatment, whereas less than one third of originally seeded Jurkat leukemia cells survived. More than 60% inhibition of cell proliferation was observed in AGS human gastric cancer cells after treatment.

TABLE 1
The effect of Gö7874 on cancer cell growth and survival.

| | | MDA- | | | | | | | | | | | |
|---|---|---|---|---|---|---|---|---|---|---|---|---|---|
| | | MB- | | | | | | | | | | | |
| | PC3 | 468 | H460 | HT29 | AGS | HepG2 | PANC-1 | Jurkat | A431 | U251 | NBT-II | Hela | KB |
| Untreated | 7.1 ± | 6.1 ± | 2.4 ± | 3.8 ± | 2.4 ± 0.3 | 4.2 ± | 2.1 ±0.3 | 8.8 ± 0.4 | 5.4 ± | 9.6 ± | 2.9 ± | 3.4 ± 0.4 | 3.8 ± 0.4 |
| (cell number × 10⁵) | 0.3 | 0.4 | 0.2 | 0.4 | | 0.4 | | | 0.4 | 0.5 | 0.4 | | |
| Treated | 0 | 0 | 0 | 0 | 0.8 ± 0.005 | 0 | 0 | 0.1 ± 0.005 | 0 | 0 | 0 | 0 | 0 |
| (cell number × 10⁵) | | | | | | | | | | | | | |

Standard errors are based on three independent experiments.

### Example 2

### The Effect of Gö6850 on Cancer Cell Growth and Survival

Cultures containing about 4×10⁴ cells were treated with or without 10 micromoles of Gö6850 and the viable cells were counted at day 6. As shown in Table 2, treated Hela human cervical cancer cells significantly reduced cell numbers and other treated cancer cells displayed 25-60% inhibition of growth in contrast to untreated cells.

TABLE 2
The effect of Gö6850 on cancer cell growth and survival.

| | | MDA- | | | | | | | | | | | |
|---|---|---|---|---|---|---|---|---|---|---|---|---|---|
| | PC3 | MB-468 | H460 | HT29 | AGS | HepG2 | PANC-1 | Jurkat | A431 | U251 | NBT-II | Hela | KB |
| Untreated | 7.1 ± | 6.1 ± | 2.4 ± | 3.8 ± | 2.4 ± | 4.2 ± 0.4 | 2.1 ± 0.3 | 8.8 ± 0.4 | 5.4 ± 0.4 | 9.6 ± 0.5 | 2.9 ± 0.4 | 3.4 ± 0.4 | 3.8 ± 0.4 |
| (cell number × | 0.3 | 0.4 | 0.2 | 0.4 | 0.3 | | | | | | | | |
| 10⁵) | | | | | | | | | | | | | |
| Treated | 2.8 ± | 4.6 ± | 1.6 ± | 2.4 ± | 1.5 ± | 2.4 ± 0.2 | 1.4 ± 0.1 | 4.1 ± 0.2 | 4.1 ± 0.2 | 7.2 ± 0.4 | 1.3 ± 0.1 | 0.3 ± 0.1 | 1.7 ± 0.1 |
| (cell number × | 0.2 | 0.2 | 0.1 | 0.2 | 0.2 | | | | | | | | |
| 10⁵) | | | | | | | | | | | | | |
| % inhibiton | 60 | 25 | 31 | 22 | 34 | 41 | 30 | 53 | 24 | 23 | 57 | * | 55 |

* indicates the inhibitor causes significant cell death, and cell number is less than originally seeded cell number.
Standard errors are based on three independent experiments.

### Example 3

### The Effect of NGIC-I on Cancer Cell Growth and Survival

Cultures containing about 4×10⁴ cells were treated with or without 10 micromoles of NGIC-I and the viable cells were counted at day 6. As shown in Table 3, KB oral cancer cells all went to apoptosis. About 50% originally seeded Jurkat leukemia cells and 75% originally seeded Hela human cervical cancer cells survived and other treated cancer cells displayed 25-82% inhibition of growth in contrast to untreated cells.

TABLE 3
The effect of NGIC-I on cancer cell growth and survival.

| | | MDA- | | | | | | | | | | | |
|---|---|---|---|---|---|---|---|---|---|---|---|---|---|
| | PC3 | MB-468 | H460 | HT29 | AGS | HepG2 | PANC-1 | Jurkat | A431 | U251 | NBT-II | Hela | KB |
| Untreated | 7.1 ± | 6.1 ± | 2.4 ± | 3.8 ± | 2.4 ± | 4.2 ± 0.4 | 2.1 ± 0.3 | 8.8 ± 0.4 | 5.4 ± 0.4 | 9.6 ± 0.5 | 2.9 ± 0.4 | 3.4 ± 0.4 | 3.8 ± 0.4 |
| (cell number × | 0.3 | 0.4 | 0.2 | 0.4 | 0.3 | | | | | | | | |
| 10⁵) | | | | | | | | | | | | | |
| Treated | 3.7 ± | 1.3x ± | 1.6 ± | 3.2 ± | 1.7 ± | 0.9 ± 0.1 | 1.2 ± 0.1 | 0.2 ± 0.1 | 2.7 ± 0.2 | 1.6 ± 0.3 | 0.9 ± 0.1 | 0.3 ± 0.1 | 0 |
| (cell number × | 0.1 | 0.2 | 0.1 | 0.1 | 0.1 | | | | | | | | |
| 10⁵) | | | | | | | | | | | | | |
| % inhibiton | 48 | 78 | 32 | 15 | 30 | 78 | 44 | * | 49 | 82 | 73 | * | * |

* indicates the inhibitor causes significant cell death, and cell number is less than originally seeded cell number.
Standard errors are based on three independent experiments.

### Example 4

### The Effect of Ro31-7549 on Cancer Cell Growth and Survival

Cultures containing about 4×10⁴ cells were treated with or without 10 micromoles of Ro31-7549 and the viable cells were counted at day 6. As shown in Table 4, all of HepG2 human liver cancer cells and KB human oral cancer cells and significant number of MDA-MB-468 human breast cancer cells and Hela human cervical cancer cells went to apoptosis upon treatment. All other treated cancer cells displayed 43-92% inhibition of growth in contrast to untreated cells.

TABLE 4
The effect of Ro31-7549 on cancer cell growth and survival.

| | | MDA- | | | | | | | | | | | |
|---|---|---|---|---|---|---|---|---|---|---|---|---|---|
| | PC3 | MB-468 | H460 | HT29 | AGS | HepG2 | PANC-1 | Jurkat | A431 | U251 | NBT-II | Hela | KB |
| Untreated | 7.1 ± | 6.1 ± | 2.4 ± | 3.8 ± | 2.4 ± | 4.2 ± 0.4 | 2.1 ± 0.3 | 8.8 ± 0.4 | 5.4 ± 0.4 | 9.6 ± 0.5 | 2.9 ± 0.4 | 3.4 ± 0.4 | 3.8 ± 0.4 |
| (cell number × | 0.3 | 0.4 | 0.2 | 0.4 | 0.3 | | | | | | | | |
| 10⁵) | | | | | | | | | | | | | |
| Treated | 2.7 ± | 0.2 ± | 0.5 ± | 1.7 ± | 1.7 ± | 0 | 1.2 ± 0.1 | 0.7 ± 0.1 | 2.8 ± 0.2 | 1.9 ± 0.3 | 0.6 ± 0.1 | 6 ± 0.1 | 0 |
| (cell number × | 0.2 | 0.05 | 0.1 | 0.1 | 0.1 | | | | | | | | |
| 10⁵) | | | | | | | | | | | | | |
| % inhibiton | 62 | * | 81 | 63 | 30 | * | 43 | 92 | 48 | 80 | 80 | * | * |

* indicates the inhibitor causes significant cell death, and cell number is less than originally seeded cell number.
Standard errors are based on three independent experiments.

### Example 5

### The Effect of Ro31-8220 on Cancer Cell Growth and Survival

Cultures containing about 4×10⁴ cells were treated with or without 10 micromoles of Ro31-8220 and the viable cells were counted at day 6. As shown in Table 5, all of AGS human gastric cancer cells, PANC-1 human pancreatic cancer cells, NBT-II human bladder cancer cells, and KB human oral cancer cells, and significant number of MDA-MB-468 human breast cancer cells, and Hela human cervical cancer cells went to apoptosis upon treatment. All other treated cancer cells displayed 60-94% inhibition of growth in contrast to untreated cells.

TABLE 5
The effect of Ro31-8220 on cancer cell growth and survival.

| | | MDA- | | | | | | | | | | | |
|---|---|---|---|---|---|---|---|---|---|---|---|---|---|
| | PC3 | MB-468 | H460 | HT29 | AGS | HepG2 | PANC-1 | Jurkat | A431 | U251 | NBT-II | Hela | KB |
| Untreated | 7.1 ± | 6.1 ± | 2.4x ± | 3.8 ± | 2.4 ± | 4.2 ± 0.4 | 2.1 ± 0.3 | 8.8 ± 0.4 | 5.4 ± 0.4 | 9.6 ± 0.5 | 2.9 ± 0.4 | 3.4 ± 0.4 | 3.8 ± 0.4 |
| (cell | 0.3 | 0.4 | 0.2 | 0.4 | 0.3 | | | | | | | | |
| number × | | | | | | | | | | | | | |
| 10⁵) | | | | | | | | | | | | | |
| Treated | 2.7 ± | 0.2 ± | 0.4 ± | 0.6 ± | 0 | 0.4 ± 0.1 | 0 | 0.5 ± 0.2 | 2.1 ± 0.2 | 1.4 ± 0.1 | 0 | 6.0 ± 0.1 | 0 |
| (cell | 0.2 | 0.05 | 0.1 | 0.1 | | | | | | | | | |
| number × | | | | | | | | | | | | | |
| 10⁵) | | | | | | | | | | | | | |
| % inhibiton | 62 | * | 85 | 85 | * | 90 | * | 94 | 60 | 85 | * | * | * |

* indicates the inhibitor causes significant cell death, and cell number is less than originally seeded cell number.
Standard errors are based on three independent experiments.

### Example 6

### The Effect of Ro31-8425 on Cancer Cell Growth and Survival

Cultures containing about 4×10⁴ cells were treated with or without 10 micromoles of Ro31-8425 and the viable cells were counted at day 6. As shown in Table 6, all of H460 human lung cancer cells, AGS human gastric cancer cells, PANC-1 human pancreatic cancer cells, Hela human cervical cancer cells, and KB human oral cancer cells, and significant number of MDA-MB-468 human breast cancer cells, Jurkat human leukemia cells, and NBT-II human bladder cancer cells went to apoptosis upon treatment. All other treated cancer cells displayed 52-90% inhibition of growth in contrast to untreated cells.

TABLE 6
The effect of Ro31-8425 on cancer cell growth and survival.

| | | MDA- | | | | | | | | | | | |
|---|---|---|---|---|---|---|---|---|---|---|---|---|---|
| | PC3 | MB-468 | H460 | HT29 | AGS | HepG2 | PANC-1 | Jurkat | A431 | U251 | NBT-II | Hela | KB |
| Untreated (cell | 7.1 ± | 6.1 ± | 2.4 ± | 3.8 ± | 2.4 ± | 4.2 ± 0.4 | 2.1 ± 0.3 | 8.8 ± 0.4 | 5.4 ± 0.4 | 9.6 ± 0.5 | 2.9 ± 0.4 | 3.4 ± 0.4 | 3.8 ± 0.4 |
| number × | 0.3 | 0.4 | 0.2 | 0.4 | 0.3 | | | | | | | | |
| 10⁵) | | | | | | | | | | | | | |
| Treated | 3 ± | 0.1 ± | 0 | 0.6 ± | 0 | 0.4 ± 0.1 | 0 | 0.3 ± 0.1 | 2.6 ± 0.2 | 1.4 ± 0.1 | 0.3 ± 0.1 | 0 | 0 |
| (cell number × | 0.2 | 0.05 | | 0.1 | | | | | | | | | |
| 10⁵) | | | | | | | | | | | | | |
| % inhibiton | 53 | * | * | 85 | * | 90 | * | * | 52 | 85 | * | * | * |

* indicates the inhibitor causes significant cell death, and cell number is less than originally seeded cell number.
Standard errors are based on three independent experiments.

### Example 7

### The Effect of Ro31-0432 on Cancer Cell Growth and Survival

Cultures containing about 4×10⁴ cells were treated with or without 10 micromoles of Ro31-0432 and the viable cells were counted at day 6. As shown in Table 7, all of cancer cells displayed 10-60% inhibition of growth in contrast to untreated cells.

TABLE 7
The effect of Ro31-0432 on cancer cell growth and survival.

| | | MDA- | | | | | | | | | | | |
|---|---|---|---|---|---|---|---|---|---|---|---|---|---|
| | PC3 | MB-468 | H460 | HT29 | AGS | HepG2 | PANC-1 | Jurkat | A431 | U251 | NBT-II | Hela | KB |
| Untreated | 7.1 ± | 6.1 ± | 2.4 ± | 3.8 ± | 2.4 ± | 4.2 ± 0.4 | 2.1 ± 0.3 | 8.8 ± 0.4 | 5.4 ± 0.4 | 9.6 ± 0.5 | 2.9 ± 0.4 | 3.4 ± 0.4 | 3.8 ± 0.4 |
| (cell | 0.3 | 0.4 | 0.2 | 0.4 | 0.3 | | | | | | | | |
| number × | | | | | | | | | | | | | |
| 10⁵) | | | | | | | | | | | | | |
| Treated | 6.0 ± | 4.3x ± | 1.3x ± | 3.0 ± | 1.7 ± | 2.4 ± 0.1 | 1.2 ± 0.1 | 1.4 ± 0.1 | 4.2 ± 0.2 | 9.2 ± 0.1 | 2.3 ± 0.1 | 1.3 ± 0.1 | 2.1 ± 0.4 |
| (cell | 0.1 | 0.2 | 0.2 | 0.3 | 0.1 | | | | | | | | |
| number × | | | | | | | | | | | | | |
| 10⁵) | | | | | | | | | | | | | |
| % inhibiton | 15 | 30 | 48 | 19 | 30 | 43 | 31 | 81 | 21 | 10 | 19 | 60 | 44 |

Standard errors are based on three independent experiments.

### PKC Inhibitors Blocked EGF-Induced Cell Transformation

3Y1 rat fibroblast cells overexpressing EGF receptors (3Y1-EGFR cells) showed a transformed morphology upon 100 ng/ml EGF treatment for 48 hours. Co-treatment of EGF with 1 micromole Gö7874 or Ro31-8425 partially blocked EGF-induced transformed morphology without detected cell apoptosis. Moreover, 3Y1-EGFR cells treated with EGF (100 ng/ml) in combination with 1 micromole Gö7874 or Ro31-8425 showed a reduction of 82% or 72% of colony number in soft agar respectively in contrast to those treated with EGF alone.

### Human and Animal Treatment

The above described test indicates clearly that Gö7874, Gö6850, Gö7612, Gö7852, Ro31-7549, Ro31-8425, Ro31-8220, Ro31-0432 and NGIC-1 and chemical compounds with similar structures have a strong apoptotic effect on a variety of human cancer cells and that it can inhibit growth of other human cancer cells. Based on the cell transformation experiments showing a blocking of the effects of EGF-induced cell transformation, Gö7874, Gö6850, Gö7612, Gö7852, Ro31-7549, Ro31-8425, Ro31-8220, Ro31-0432 and NGIC-1 and chemical compounds with similar structure also can be used as a cancer preventive reagent. It could be applied in many of the well-known methods currently used for chemotherapeutic treatment. For example, it may be injected directly into tumors, injected into the blood stream or body cavities or taken orally or applied through the skin with patches. The dose chosen should be sufficient to constitute effective treatment but not so high as to cause unacceptable side effects. The state of the cancer and the health of the patient should preferably be closely monitored during and for a reasonable period after treatment.

While the above examples describe the effectiveness of Gö7874, Gö6850, Gö7612, Gö7852, Ro31-7549, Ro31-8425, Ro31-8220, Ro31-0432 and NGIC-1 and chemical compounds with similar structures as chemotherapeutic treatment for various cancers, they are not intended as a limitation of the invention. The chemical was effective in killing or reducing the growth rate of all cancer cells tested. Therefore, it should be obvious that it would be effective as a treatment to cancer cells, which were not tested, that are associated with increased levels of PKC α expression and/or activity such as, nasopharyngeal carcinoma, human renal cell carcinomas, tumor derived from endocrine glands such as the pituitary gland and the thyroid gland, esophagus cancer, and melanoma. Derivatives of Gö7874, Gö6850, Gö7612, Gö7852, Ro31-7549, Ro31-8425, Ro31-8220, Ro31-0432 and NGIC-1 that are effective in inhibiting PKC α should also be effective as a cancer treatment. Also, since Gö7874, Gö6850, Gö7612, Gö7852, Ro31-7549, Ro31-8425, Ro31-8220, Ro31-0432 and NGIC-1 and chemical compounds with similar structures prevent cell transformation, it should also work as a preventative drug, especially for people having a high risk for particular cancers.

Although the present invention has been described with reference to certain exemplary embodiments thereof, it will be understood by those skilled in the art that a variety of modifications and variations may be made to the present invention without departing from the spirit or scope of the present invention defined in the appended claims, and their equivalents.

What is claimed is:

1. A method of treating or preventing a cancer, comprising:
administering to a mammal a chemical targeted to PKC α, said chemical being a compound represented by one of the following formulae or its derivative: 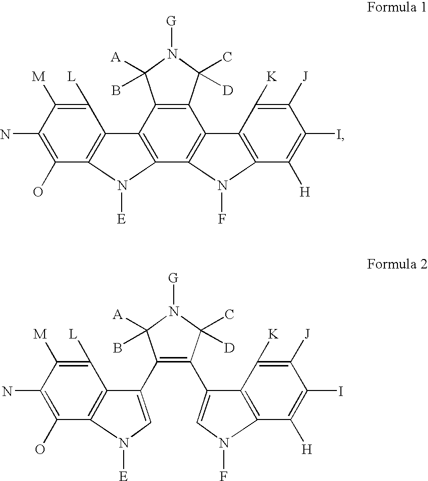
wherein each or a combination of two of A, B, C, D, E, F, G, H, I, J, K, L, M, N, and O is independently selected from the group consisting of hydrogen, oxygen, methyl, ethyl, propyl, isopropyl, carboxymethyl, 2-carboxyethyl, 3-carboxypropyl, a straight or branched alkyl group, a straight or branched substituted alkyl group, a straight or branched azidoalkyl, carboxyalkyl, amidinothioalkyl, amidinoalkyl, (2-nitroguanidino) alkyl group, cyanoalkyl, and —(CH₂)₂—CO—NX wherein X is hydrogen, alkyl, or benzyl, and E is not methyl when A and B is an oxygen, each of C, D, G, H and I is hydrogen, and the compound represented by Formula 1 is not 12-(2-Cyanoethyl)-6,7,12,13-tetrahydro-13-methyl-5-oxo-5H-indolo[2,3-a]pyrrolo[3,4-c]carbazole, 12-(3-Aminopropyl)-5,6,7,12,13-pentahydro-indolo[2,3,-a]pyrrolo [3,4-c]carbazole, or 12-(3-Aminopropyl)-5,6,7,12,13-pentahydro-indolo[2,3,-a]pyrrolo[3,4-c]carbazole hydrochloride; and
monitoring said mammal to determine state of said cancer.

2. A method of claim 1, wherein said chemical is selected from the group consisting of a salt and a regioisomeric mixture of at least one of the compounds represented by Formula 1 and Formula 2.

3. A method of claim 1, wherein said chemical is selected from a group consisting of Gö7874, Gö6850, Gö7612, Gö7852, Ro31-7549, Ro31-8425, Ro31-8220, Ro31-0432, NGIC-I, a derivative thereof, and a salt thereof.

4. A method of claim 1, wherein said chemical is 5,7(6H)-dione,13-[3-(dimethylamino)-2-hydroxypropyl]-12,13-dihydro-3-methoxy-12-methyl-5H-Indolo[2,3 -a]pyrrolo[3,4-c]carbazole, monohydrochloride.

5. A method of claim 1, wherein said chemical is 2-[1-(3-Dimethylaminopropyl)-1H-indol-3-yl]-3-(1H-indol-3-yl)-maleimide.

6. A method of claim 1, wherein said chemical is 12-propanenitrile,5,6,7,13-tetrahydro-5-oxo-12H-Indolo[2,3-a]pyrrolo[3,4-c]carbazole.

7. A method of claim 1, wherein said chemical is 5-one, 12-[3-(dimethylamino)-2-hydroxypropyl]-6,7,12,13-tetrahydro-13-methyl-5H-Indolo[2,3-a]pyrrolo[3,4-c]carbazol.

8. A method of claim 1, wherein said chemical is 12-propanenitrile,5,6,7,13-tetrahydro-9-methoxy-13-methyl-5,7-dioxo-12H-Indolo[2,3-a]pyrrolo[3,4-c]carbazole.

9. A method of claim 1, wherein said chemical is 2-[1-3(Aminopropyl)indol-3-yl]-3(1-methyl-1H-indol-3-yl)maleimide, Acetate.

10. A method of claim 1, wherein said chemical is 3-[1-[3-(Amidinothio)propyl-1H-indol-3-yl]-3-(1-methyl-1H-indol-3-yl)maleimide.

11. A method of claim 1, wherein said chemical is 2-[8-(Aminomethyl)-6,7,8,9-tetrahydropyrido[1,2-a]indol-3-yl]-3-(1-methyl-1H-indol-3-yl)maleimide, HCl.

12. A method of claim 1, wherein said chemical is 3-[1-[3-(Amidinothio)propyl-1H-indol-3-yl]-3-(1-methyl-1H-indol-3-yl)maleimide.

13. A method of claim 1, wherein said mammal is a human.

14. A method of claim 1, wherein said cancer is selected from the group consisting of breast cancer, leukemia, lymphoma, lung cancer, skin cancer, melanoma, prostate cancer, gastric cancer, colon cancer, rectal cancer, brain tumor, liver cancer, cervical cancer, bladder cancer, oral cancer, pancreatic cancer, renal cancer, nasopharyngeal cancer, esophagus cancer, and tumor derived from endocrine glands, the pituitary gland carcinoma and the thyroid gland carcinoma.

15. A method of claim 1, wherein said chemical is administered directly into a tumor or said mammal's blood stream, administered orally, administered through the skin of said mammal, or administered in combination with radiation therapy.

16. A method of treating or preventing cancer, comprising:
administering to a mammal a chemical selected from the group consisting of a compound represented by one of the following formulae, a derivative of the compound, a salt of the compound, a regioisomer of the compound, and their mixture: 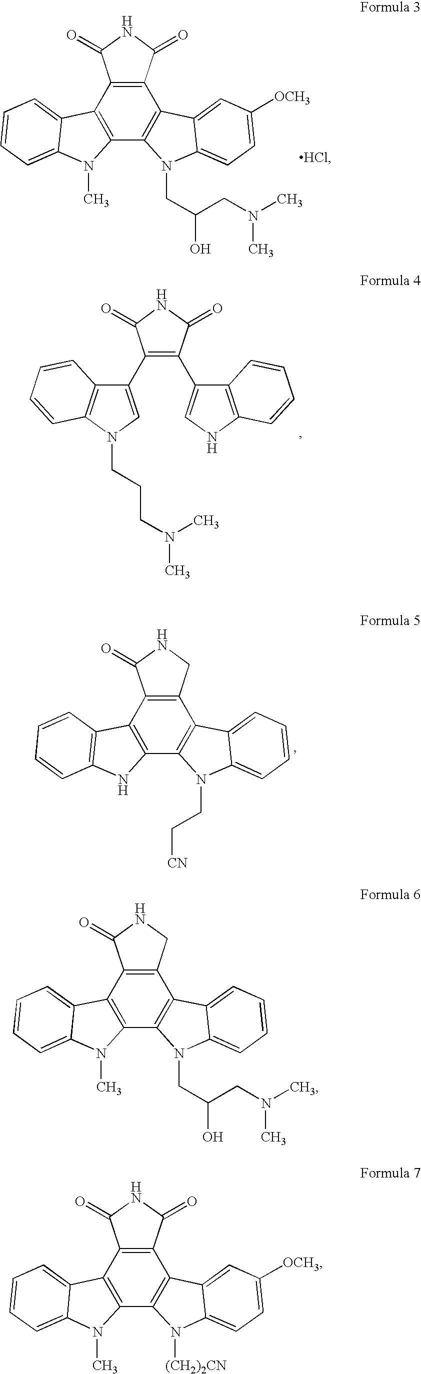 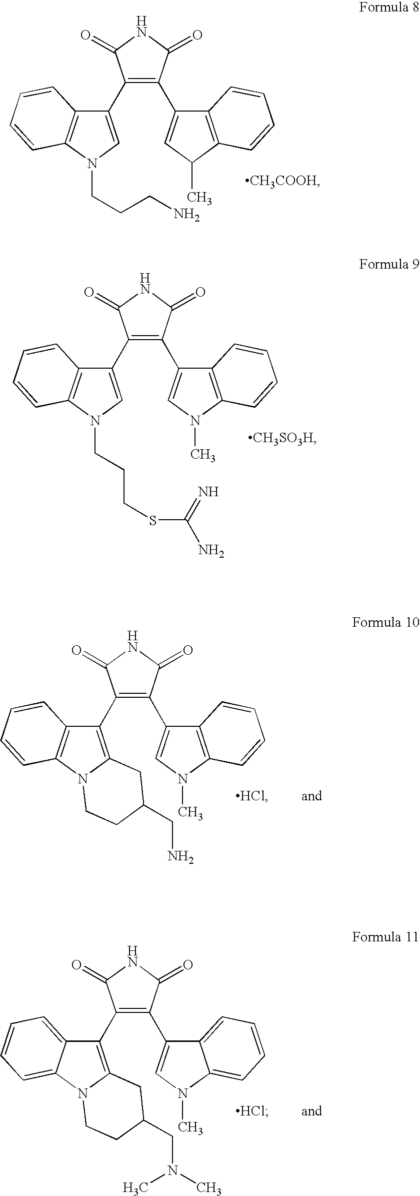
monitoring said mammal to determine state of said cancer.

17. A method of claim 16, wherein said cancer is selected from the group consisting of breast cancer, leukemia, lymphoma, lung cancer, skin cancer, melanoma, prostate cancer, gastric cancer, colon cancer, rectal cancer, brain tumor, liver cancer, cervical cancer, bladder cancer, oral cancer, pancreatic cancer, renal cancer, nasopharyngeal cancer, esophagus cancer, tumor derived from endocrine glands, the pituitary gland carcinoma, and the thyroid gland carcinoma.

18. A method of utilizing a compound selected from the group consisting of 5,7(6H)-dione, 13-[3-(dimethylamino)-2-hydroxypropyl]-12,13-dihydro-3-methoxy-12-methyl-5H-Indolo[2,3-a]pyrrolo[3,4-c]carbazole, monohydrochloride, 2-[1-(3-Dimethylaminopropyl)-1H-indol-3-yl]-3-(1H-indol-3-yl)-maleimide, 12-propanenitrile,5,6,7,13-tetrahydro-5-oxo-12H-Indolo[2,3-a]pyrrolo[3,4-c]carbazole, 5-one, 12-[3-(dimethylamino)-2-hydroxypropyl]-6,7,12,13-tetrahydro-13-methyl-5H-Indolo[2,3-a]pyrrolo[3,4-c]carbazol, 12-propanenitrile, 5,6,7,13-tetrahydro-9-methoxy-13-methyl-5,7-dioxo-12H-Indolo[2,3-a]pyrrolo[3,4-c]carbazole, 2-[1-3(Aminopropyl)indol-3-yl]-3(1-methyl-1H-indol-3-yl)maleimide, Acetate, 3-[1-[3-(Amidinothio)propyl-1H-indol-3-yl]-3-(1-methyl-1H-indol-3-yl)maleimide, 2-[8-(Aminomethyl)-6,7,8,9-tetrahydropyrido[1,2-a]indol-3-yl]-3-(1-methyl-1H-indol-3-yl)maleimide, HCl, 3-[1-[3-(Amidinothio)propyl-1H-indol-3-yl]-3-(1-methyl-1H-indol-3-yl)maleimide, a derivative thereof, and a salt thereof, the method comprising:
utilizing the compound for treating or preventing cancer associated with increased levels of PKC α expression or activity.

19. A method of claim 18, wherein said cancer is selected from the group consisting of breast cancer, leukemia, lymphoma, lung cancer, skin cancer, melanoma, prostate cancer, gastric cancer, colon cancer, rectal cancer, brain tumor, liver cancer, cervical cancer, bladder cancer, oral cancer, pancreatic cancer, renal cancer, nasopharyngeal cancer, esophagus cancer, tumor derived from endocrine glands, the pituitary gland carcinoma, and the thyroid gland carcinoma.
